(19) United States
(12) Patent Application Publication
(10) Pub. No.: US 2007/0049936 A1
(43) Pub. Date: Mar. 1, 2007

(54) ALIGNMENT INSTRUMENT FOR DYNAMIC SPINAL STABILIZATION SYSTEMS

(76) Inventors: Dennis Colleran, North Attleboro, MA (US); Arnold Oyola, Northborough, MA (US); Michael Perriello, Hopedale, MA (US); Sally Carter, North Attleboro, MA (US); Carolyn Rogers, Frisco, TX (US)

Correspondence Address: CARR LLP (IST), 670 FOUNDERS SQUARE, 900 JACKSON STREET, DALLAS, TX, 75202, US

(21) Appl. No.: 11/467,798

(22) Filed: Aug. 28, 2006

Related U.S. Application Data
(60) Provisional application No. 60/711,812, filed on Aug. 26, 2005.

(51) Int. Cl. A61F 2/30

(52) U.S. Cl. 606/61

(57) ABSTRACT

Several aspects of alignment systems and methods used in spinal surgery are disclosed. For instance, in one aspect, there is disclosed an alignment instrument comprising a first alignment member for coupling to a spinal stabilization system at one location, a second alignment member for coupling to the spinal stabilization system at a second location, and a linkage assembly for coupling the first alignment member to the second alignment member.

### CROSS-REFERENCES AND CLAIM OF PRIORITY

This application claims priority to U.S. Provisional Patent Application Ser. No. 60/711,812, filed on Aug. 26, 2005, which is incorporated herein by reference.

This application is related to commonly assigned U.S. Provisional Application Ser. No. 60/786,898, entitled “FULL MOTION SPHERICAL LINKAGE IMPLANT SYSTEM,” filed Mar. 29, 2006; U.S. Provisional Application Ser. No. 60/793,829, entitled “MICRO-MOTION IMPROVEMENTS,” filed on Mar. 29, 2006; U.S. Provisional Application Ser. No. 60/831,879, entitled “LOCKING ASSEMBLY,” filed on Jul. 19, 2006; U.S. Utility Application Serial No. 11/443,236, entitled, “SYSTEM AND METHOD FOR DYNAMICAL SKELETAL STABILIZATION,” filed on May 30, 2006; U.S. Provisional Application Ser. No. 60/692,943, entitled “SPHERICAL MOTION DYNAMIC SPINAL STABILIZATION DEVICE,” filed Jun. 22, 2005; International Patent Application No. PCT/US05/27996, entitled “SYSTEM AND METHOD FOR DYNAMIC SKELETAL STABILIZATION,” filed Aug. 8, 2005, and to commonly assigned U.S. patent application Ser. No. 10/690,211, entitled “SYSTEM AND METHOD FOR STABILIZING INTERNAL STRUCTURES,” filed Oct. 21, 2003, all of which are incorporated herein by reference.

### FIELD OF THE INVENTION

This disclosure relates to skeletal stabilization and, more particularly, to aligning dynamic stabilization systems for the stabilization of human spines.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include horizontal movement (bending either forward/anterior or aft/posterior), rolling movement (bending to either left or right side) and vertical movement (twisting of the shoulders relative to the pelvis).

In flexing about the horizontal axis into flexion (bending forward or in an anterior direction) and extension (bending backward or in a posterior direction), vertebrae of the spine must rotate about the horizontal axis to various degrees. The sum of all such movement about the horizontal axis produces the overall flexion or extension of the spine. For example, the vertebrae that make up the lumbar region of the human spine move through roughly an arc of 15° relative to adjacent or neighboring vertebrae. Vertebrae of other regions of the human spine (e.g., the thoracic and cervical regions) have different ranges of movement. Thus, if one were to view the posterior edge of a healthy vertebra, one would observe that the edge moves through an arc of some degree (e.g., of about 15° in flexion and about 5° in extension if in the lumbar region) centered about a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together while the anterior edges move farther apart, thereby compressing the posterior of the spine. During flexion and extension the vertebrae move in horizontal relationship to each other providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through an arc of around 10° relative to neighbor vertebrae throughout right and left lateral bending.

Rotational movement about a vertical axis relative is also natural in the healthy spine. For example, rotational movement can be described as the clockwise or counter-clockwise twisting rotation of the vertebrae during a golf swing.

In a healthy spine, the inter-vertebral spacing between neighboring vertebrae is maintained by a compressible and somewhat elastic disc. The disc serves to allow the spine to move about the various axes of rotation and through the various arcs and movements required for normal mobility. The elasticity of the disc maintains spacing between the vertebrae during flexion and lateral bending of the spine, thereby allowing room or clearance for compression of neighboring vertebrae. In addition, the disc allows relative rotation about the vertical axis of neighboring vertebrae, allowing twisting of the shoulders relative to the hips and pelvis. A healthy disc further maintains clearance between neighboring vertebrae, thereby enabling nerves from the spinal chord to extend out of the spine between neighboring vertebrae without being squeezed or impinged by the vertebrae.

In situations where a disc is not functioning properly, the inter-vertebral disc tends to compress, thereby reducing inter-vertebral spacing and exerting pressure on nerves extending from the spinal cord. Various other types of nerve problems may be experienced in the spine, such as exiting nerve root compression in the neural foramen, passing nerve root compression, and enervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to one another in order to maintain space for the nerves to exit without being impinged upon by movements of the spine.

In one such procedure, screws are embedded in adjacent vertebrae pedicles and rigid rods or plates are then secured between the screws. In such a situation, the pedicle screws press against the rigid spacer that serves to distract the degenerated disc space, thereby maintaining adequate separation between the neighboring vertebrae to prevent the vertebrae from compressing the nerves. Although the foregoing procedure prevents nerve pressure due to extension of the spine, when the patient then tries to bend forward (putting the spine in flexion), the posterior portions of at least two vertebrae are effectively held together. Furthermore, the lateral bending or rotational movement between the affected vertebrae is significantly reduced due to the rigid connection of the spacers. Overall movement of the spine is reduced as more vertebrae are distracted by such rigid spacers. This type of spacer not only limits the patient's movements, but also places additional stress on other portions of the spine, such as adjacent vertebrae without spacers, often leading to further complications at a later date.

In other procedures, dynamic fixation devices are used. However, conventional dynamic fixation devices may not facilitate lateral bending and rotational movement with respect to the fixated discs. This can cause further pressure on the neighboring discs during these types of movements, which over time may cause additional problems in the neighboring discs. Furthermore, alignment of such dynamic fixation devices to enable a relatively natural range of motion while restricting undesirable motion is often difficult.

Accordingly, improvements are needed in alignment instruments for aligning dynamic systems that approximate and enable a fuller range of motion while providing stabilization of a spine.

### SUMMARY

Several aspects of alignment systems and methods used in spinal surgery are disclosed. For instance, in one aspect, there is disclosed an alignment instrument comprising a first alignment member for coupling to a spinal stabilization system at one location, a second alignment member for coupling to the spinal stabilization system at a second location, and a linkage assembly for coupling the first alignment member to the second alignment member.

### BRIEF DESCRIPTION OF THE DRAWINGS

Aspects of the present disclosure are best understood from the following detailed description when read with the accompanying figures. It is emphasized that various features may not be drawn to scale. In fact, the dimensions of the various features may be arbitrarily increased or reduced for clarity of discussion.

FIG. 1A is an isometric view of a portion of a spine.

FIG. 1B is a perspective view of one embodiment of a dynamic stabilization system.

FIG. 2 is a simplified diagrammatic perspective view of the dynamic stabilization system of FIG. 1B.

FIGS. 3A and 3B are perspective views of the simplified dynamic stabilization system of FIG. 2 in a generally neutral position and in flexion/extension, respectively.

FIGS. 4A and 4B are perspective views of the simplified dynamic stabilization system of FIG. 2 in a generally neutral position and in lateral bending, respectively.

FIGS. 5A and 5B are perspective views of the simplified dynamic stabilization system of FIG. 2 in a generally neutral position and in rotation, respectively.

FIG. 6 is a perspective view of one embodiment of an alignment instrument in use with a dynamic stabilization system, such as the dynamic stabilization system of FIG. 1B.

FIGS. 7A and 7B are perspective views of another embodiment of an alignment instrument in use with a dynamic stabilization system.

FIG. 8 is a perspective view of yet another embodiment of an alignment instrument in use with a dynamic stabilization system.

FIG. 9 is a flow chart of one embodiment of a method for substantially aligning a dynamic stabilization system.

FIGS. 10A-10E are photographs illustrating the method of FIG. 9.

FIG. 11 is a flow chart of another embodiment of a method for substantially aligning a dynamic stabilization system.

FIG. 12 is a perspective view of another embodiment of an alignment instrument for use with a dynamic stabilization system.

FIG. 13 is a side view of the alignment instrument of FIG. 12.

FIG. 14 is a flow chart of yet another embodiment of a method for substantially aligning a dynamic stabilization system.

### DETAILED DESCRIPTION

It is to be understood that the following disclosure provides many different embodiments, or examples, for implementing different features of the invention. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

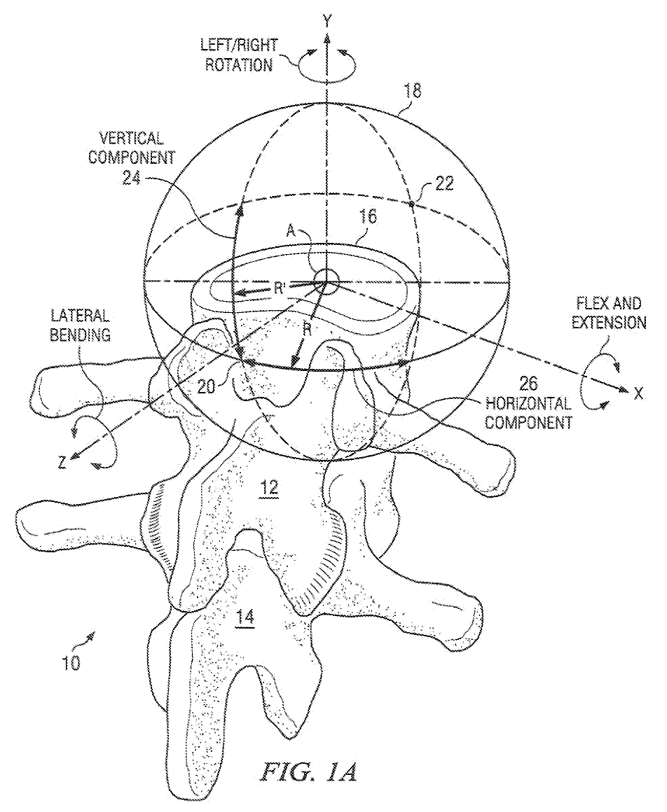

Referring to FIG. 1A, a portion of a spine 10 is shown in an isometric view. The spine portion 10 includes a vertebra 12 and a lower vertebra 14. In an actual spine, an intervertebral disc (not shown) may be located above a vertebral plate 16 of the vertebra 12, but is omitted for clarity. Furthermore, an upper adjacent vertebra (similar to vertebra 12) may be positioned above the intervertebral disc, but this upper adjacent vertebra is also omitted for clarity. In the present example, imaginary “x”, “y”, and “z” axes are superimposed upon the spine portion 10. The intersection of the axes may be defined to be a center point “A” which, for purposes of this discussion, is positioned above the vertebral plate 16 within the intervertebral space.

Natural spine motion may be modeled in relation to the x, y, and z axes. As previously discussed, flexion or extension movement may be modeled as a rotation of the vertebra about the x-axis. Lateral bending (bending towards the right or left) may be modeled as rotation about the z-axis. Rotation (twisting the torso in relation to the legs) may be modeled as rotation about the y-axis. Thus, the relative natural movement of the vertebrae of the spine 10 may occur in three dimensions with respect to the three illustrated axes.

Generally, a dynamic stabilization system that may be used to stabilize the vertebra 12 with respect to an upper vertebra (not shown) facing the endplate 16 may be oriented so that various axes of the dynamic stabilization system are aligned with a center or centroid of rotation (i.e., the center point labeled “A”). To facilitate such alignment, an alignment system may be used that enables a surgeon to align the various axes of the dynamic stabilization system with the center point “A”.

As will be described later in greater detail, the alignment instrument may be used to align the dynamic stabilization system so that the dynamic stabilization system moves along the surface of an imaginary three dimensional curved body, such as a sphere or ellipsoid. For discussion purposes, a sphere 18 is shown superimposed upon spine portion 10. The center of the sphere 18 is at the center of rotation “A.” The dynamic stabilization system may be aligned so that a point on an upper vertebra (not shown) may move in relation to a corresponding point on the vertebra 12 by following a path that is generally restricted to the surface of the sphere 18 (or other three dimensional shape).

For instance, using the sphere 18 as an example, assume a path has a starting point at point 20 which is on the surface of the sphere 18. Further assume that the path has an ending point 22 which is also on the surface of the sphere 18. Thus, it can be seen that the path between point 20 and point 22 that follows the surface of the sphere 18 has a vertical component 24 and a horizontal component 26. Movement that is restricted to the vertical curved component 24 is considered to be two dimensional movement or rotation about the x-axis. Movement that is restricted to the horizontal component 26 is also two dimensional movement, but represents rotation about the y-axis. The combination of the vertical curved component and the horizontal curved component represents three-dimensional movement about the center of rotation “A”.

If the path between points is restricted to the surface of a sphere, the path will have a constant radius of curvature “R” with respect to the center of rotation “A.” In some embodiments, the horizontal component 26 may have a radius of curvature R and the vertical component 24 may have a radius of curve R′. Thus, if the radii of curvature R equals R′ and they have the same center of rotation, the path would be on a sphere as illustrated in FIG. 1A. However, if R′ does not equal R, then the imaginary three dimensional curved body may be an ellipsoid or another three dimensional surface.

The desired location of the center point “A” with respect to the endplate 16 may vary depending on factors such as the patient's particular spinal structure and the desired result of the operation, and the surgeon may need to position the center point at a particular location within a range of possible locations. Such positioning may entail moving the center point “A” within a two or three dimensional space along any or all of the x-axis, y-axis, and z-axis. Accordingly, the alignment instrument may enable the surgeon to position the center point “A” where desired (within limitations imposed by the spinal structure and/or the alignment instrument itself) and to maintain the alignment of the dynamic stabilization system with the center point when the center point is repositioned.

It is understood that, although the center point “A” may represent the center of rotation of a sphere as illustrated in FIG. 1A, it is not limited to a single discrete point or a sphere. For example, the center of rotation may be a spherical or ellipsoidal area around which a dynamic stabilization system may rotate. Accordingly, the term “center of rotation” as used in the present disclosure is for purposes of illustration and is not limited to a single discrete point or to rotation around a spherical body.

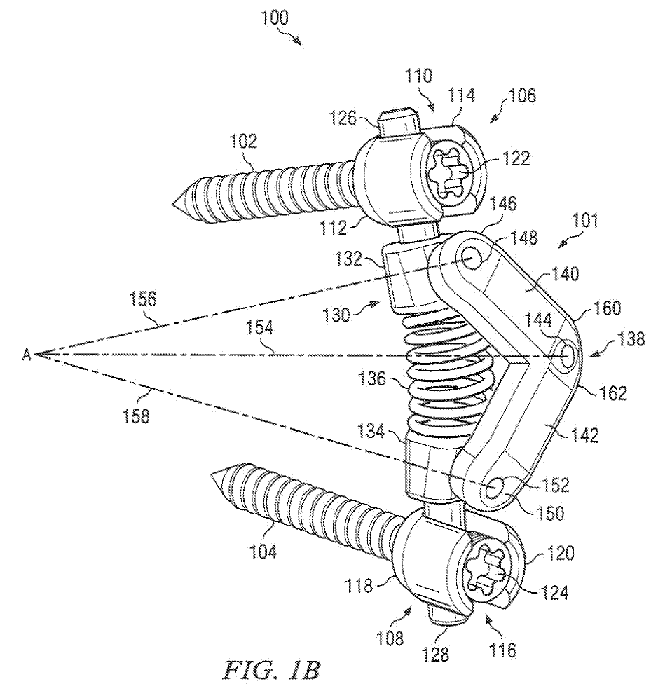

Referring to FIGS. 1B and 2, an embodiment of a dynamic stabilization system 100 having a spinal stabilization device 101 will now be described. It is understood that the particular dynamic stabilization system 100 and device 101 described herein are for purposes of example only, and that the various embodiments of alignment instruments disclosed in the present application may be used with spinal stabilization systems and devices other than those illustrated in FIG. 1B. Furthermore, it is understood that the dynamic stabilization system 100 may include multiple spinal stabilization devices.

The dynamic stabilization system 100 may be designed to permit a limited degree of movement between neighboring vertebrae in flexion/extension, lateral bending, and rotation directions, while restraining the degree of movement generally along an imaginary shell (e.g., a three dimensional shape) about a center of rotation “A”. In the present example, the shell is generally spherical and the center of rotation may lie at the origin of the spherical shell. However, it is understood that the shell may be another shape, such as an ellipsoid. Accordingly, the present disclosure is not limited to a center of rotation within a spherical shell.

The dynamic stabilization system 100 may include bone anchors 102 and 104, which may be coupled to polyaxial heads 106 and 108, respectively. The polyaxial head 106 may include a slot 110 formed by sidewalls 112 and 114, and the polyaxial head 108 may include a slot 116 formed by sidewalls 118 and 120. An interior portion of each sidewall 112, 114, 116, and 118 may be threaded to receive a locking cap 122 or 124. The slots 110 and 116 may be configured to receive an extension (e.g., a rod) 126 and 128, respectively, that may form part of the spinal stabilization device 101. Each polyaxial head 106 and 108 may move relative to a longitudinal axis of their respective bone anchor until locked down by tightening the respective locking caps 122 and 124 against extensions 126 and 128. The dynamic stabilization system 100 may also include the spinal stabilization device 101, which may be coupled to the polyaxial heads 106 and 108.

The spinal stabilization device 101 may include extensions 126 and 128. In the present embodiment, the extensions 126 and 128 may be coupled by a flexible support column 130. The flexible support column 130 may include a collar 132 and a collar 134 with a resilient member 136, such as a coil spring, positioned therebetween.

The spinal stabilization device 101 may also include an elbow 138 having an upper member 140 and a lower member 142 pivotally interconnected at pivot 144. The distal end 146 (relative to pivot 144) of upper member 140 may be pivotally connected to collar 132 at pivot 148 and the distal end 150 (relative to pivot 144) of lower member 142 may be pivotally connected to collar 134 at pivot pin 152.

In the present embodiment, the elbow 138 may be designed so that an axis passing longitudinally through each pivot pin 144, 148, and 152 (e.g., axes 154, 156, and 158, respectively) intersects a center of rotation “A”. It is understood that factors such as the length of the upper member 140 and lower member 142, the angle of the pivot pins 144, 148, and 152, and an amount of curvature in each of the upper and lower members, may alter the location of the center of rotation “A”. Due to the design of the elbow 138, the spinal stabilization device 101 may allow flexion/extension, rotation, and/or lateral bending while the axes 154, 156, and 158 maintain their intersection with the center of rotation “A”.

In operation, bone anchors 102 and 104 may be attached to respective vertebrae (not shown) by screwing threaded portions of each anchor into the bone of the vertebrae. The polyaxial heads 106 and 108 may be coupled to their respective bone anchors 102 and 104 either before or after the bone anchors are inserted in the vertebrae. The first and second extensions 126 and 128 may be placed into the slots 110 and 116. At this point, the polyaxial heads 106 and 108 may move with respect to the bone anchors 102 and 104, respectively, to allow for proper positioning of the spinal stabilization device 101. Once in position, locking caps 122 and 124 may be tightened to lock the extensions 126 and 128 into position. This may also force extensions 126 and 128 into bone anchors 102 and 104, respectively, thereby locking polyaxial heads 106 and 108 into position.

In certain embodiments, a damping element (e.g., the spring 136) may be installed between the vertebrae in a somewhat compressed condition to provide a vertical force for at least partially unloading an inter-vertebral disc, and to allow limited axial and bending movement between the neighboring vertebrae. In some embodiments, a partial disc replacement (PDR) element (not shown) may be used to provide interior support between the vertebrae.

Referring to FIG. 2, one embodiment of a simplified spinal stabilization device, such as the spinal stabilization device 101 of FIG. 1B, is illustrated. In the present example, the distal end 146 of upper member 140 is bent about an axis longitudinal to the upper member and about an axis perpendicular to the upper member, so that, when the elbow 138 is positioned in its approximately middle position (e.g., as depicted in FIG. 1B), the axis 156 of pivot 148 points downwardly and inwardly towards the center of rotation “A”. The distal end 150 of lower member 142 may be similarly bent about an axis longitudinal to the lower member and about an axis perpendicular to the lower member, so that the axis 158 of pivot 152 points upwardly and inwardly towards the same point “A.” Proximal ends 160 and 162 of upper and lower members 140 and 142, respectively, may also be shaped so that the axis 154 of pivot 144 coupling the proximal ends also points inwardly towards the same point “A”.

Bone anchors 102 and 104 may be installed in vertebral bodies (not shown) such that point “A” may be located as illustrated, for example, in FIG. 1B. Because the axis of each of the pivots 144, 146, and 152 point generally towards the same center of rotation “A”, the elbow 138 may restrict movement of the pivots about an imaginary spherical shell having a center of rotation at “A” as the vertebrae move relative to one another in flexion/extension, rotation, and/or lateral bending. This may restrict movement of the anchors 102 and 104, and hence the vertebrae themselves, to movement about the center of rotation “A”. In some embodiments, this spherical movement about a center of rotation may mimic a natural motion of adjacent vertebrae as they move generally about the center of a healthy, natural disc when cushioned by the disc.

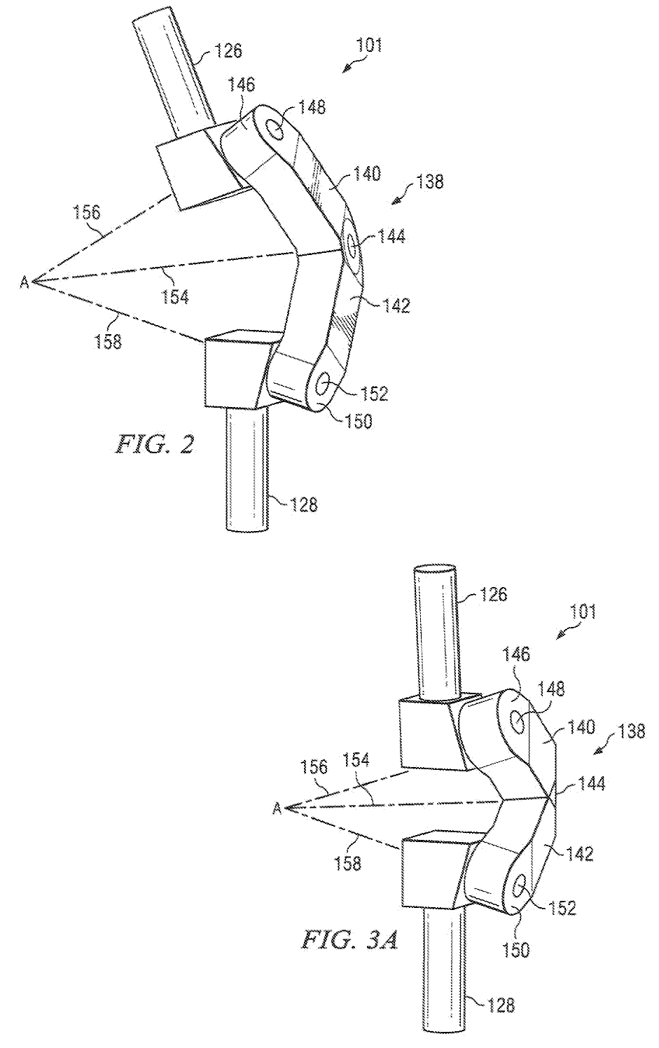

Referring to FIGS. 3A and 3B, an embodiment of the simplified spinal stabilization device 101 of FIG. 2 diagrammatically illustrates the generally spherical movement of the pivots 144, 146, and 152 about the center of rotation “A” during flexion/extension. More specifically, FIG. 3A illustrates the position of the upper and lower members 140 and 142 in a generally middle or “neutral” position and FIG. 3B illustrates the position of the upper and lower members after flexion/extension, as would occur when a person bends forward. As illustrated, the axes 154, 156, and 158 intersect the center of rotation “A” in either position.

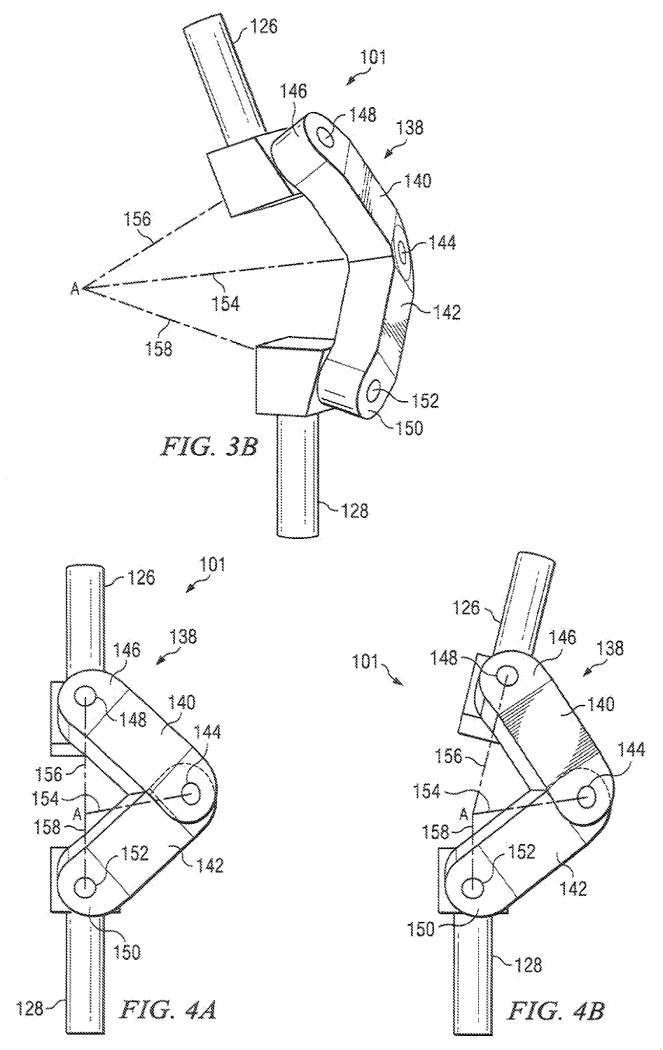

Referring to FIGS. 4A and 4B, an embodiment of the simplified spinal stabilization device 101 of FIG. 2 diagrammatically illustrates the generally spherical movement of the pivots 144, 146, and 152 about the center of rotation “A” during lateral bending. More specifically, FIG. 4A illustrates the position of the upper and lower members 140 and 142 in a generally middle or “neutral” position and FIG. 4B illustrates the position of the upper and lower members after bending to the right and slightly forward.

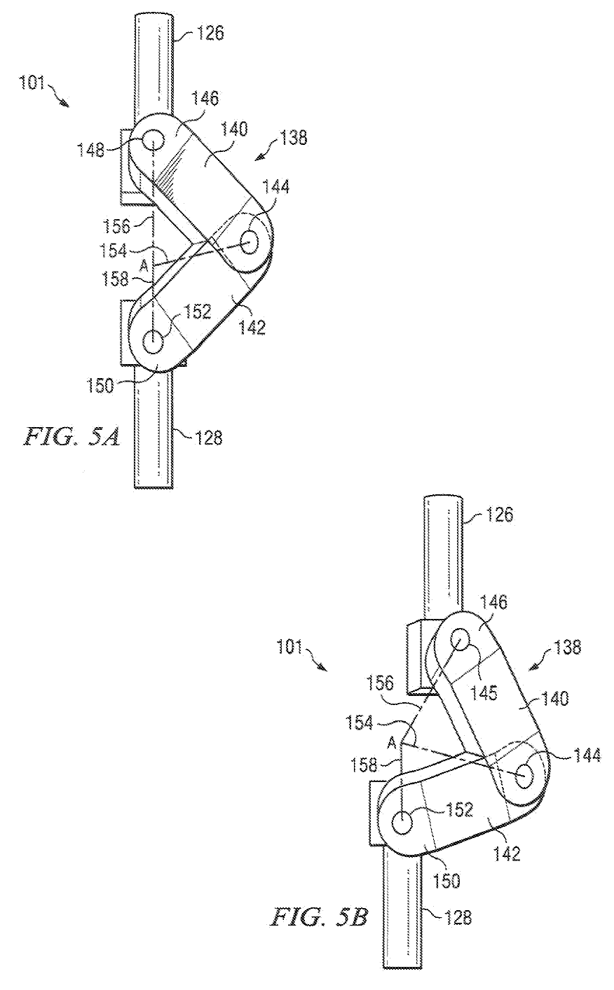

Referring to FIGS. 5A and 5B, an embodiment of the simplified spinal stabilization device 101 of FIG. 2 diagrammatically illustrates the generally spherical movement of the pivots 144, 146, and 152 about the center of rotation “A” during rotation. More specifically, FIG. 5A illustrates the position of the upper and lower members 140 and 142 in a generally middle or “neutral” position and FIG. 5B illustrates the position of the upper and lower members after clockwise rotation, as would occur when a person turns to the right.

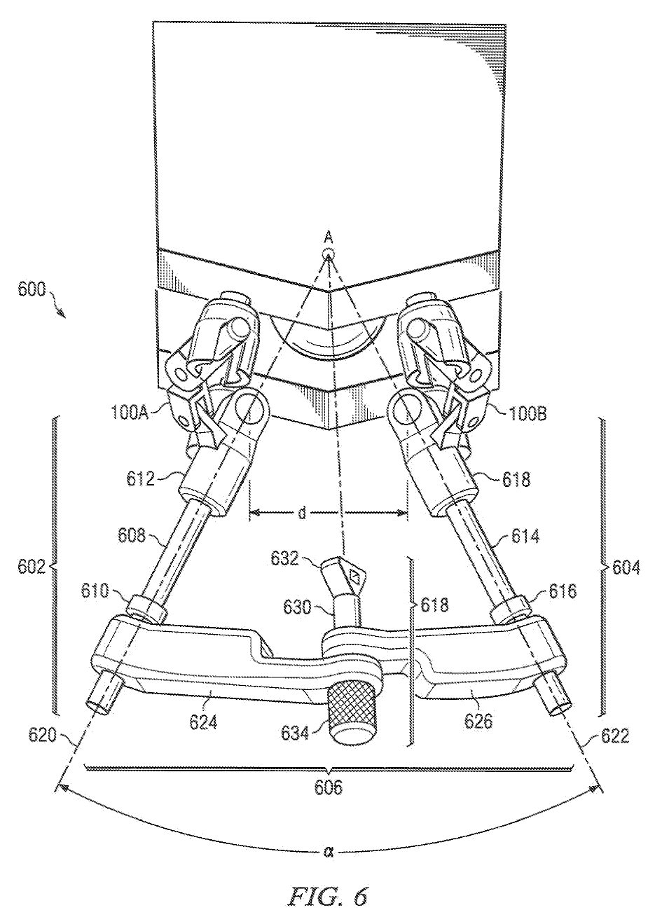

Referring to FIG. 6, in one embodiment, an alignment instrument 600 may be used to align one or more dynamic stabilization systems 100A and 100B (e.g., the dynamic stabilization system 100 of FIG. 1B) to a center of rotation “A”. As described previously, portions of the dynamic stabilization system 100 may be configured to rotate around the center of rotation “A”. As illustrated in FIG. 6, the alignment instrument 600 may attach to the dynamic stabilization systems 100A and 100B and may be used to alter the position of one or both of the dynamic stabilization systems prior to locking the dynamic stabilization systems into position.

In the present example, the alignment instrument 600 may include alignment members 602 and 604 that may be coupled by a linkage assembly 606. The alignment member 602 may include a shaft 608 having a collar 610 near a proximal end thereof and a coupler 612 at a distal end thereof. The alignment member 604 may include a shaft 614 having a collar 616 near a proximal end thereof and a coupler 618 at a distal end thereof. Each coupler 612 and 618 may be configured to removably couple to a portion of a spinal stabilization system, such as a polyaxial head. The alignment members 602 and 604 may each have a longitudinal axis 620 and 622, respectively. In the present example, the longitudinal axes 620 and 622 may extend from the proximal end of each alignment member 602 and 604 to the distal ends, and may intersect the center of rotation “A”.

The linkage assembly 606 may include arms 624 and 626 that may be pivotally coupled to one another at a proximal end of each arm. The arm 624 may be coupled to alignment member 602 and the arm 626 may be coupled to alignment member 604. In the present example, a distal end of each arm 624 and 626 may include a bore therethrough for receiving the proximal ends of the alignment members 602 and 604, respectively. More specifically, the bores may each have a longitudinal axis that may intersect the center of rotation “A”. For example, the axes of the bores may coincide with the axes 620 and 622 of the alignment members 602 and 604. The collars 610 and 616 may prevent the arms 624 and 626, respectively, from movement in the direction of the distal ends of the alignment members 602 and 604.

The linkage assembly 606 may also include a guide mechanism, such as guide pin assembly 628. In this embodiment, the guide pin assembly 628 may include a shaft 630 having a foot 632 at a distal end thereof and a knob 634 at a proximal end thereof. The foot 632 may be placed proximal to or in contact with an outer tissue layer of a patient, and the shaft 630 may be used for alignment purposes using, for example, fluoroscopy techniques. The knob 634 may be used to adjust the relative positions of the arms 624 and 626 and, accordingly, the corresponding alignment members 602 and 604.

As described previously, linkage assembly 606 and corresponding alignment members 602 and 604 may be designed to point towards the common center of rotation “A”. It is understood that the common center of rotation “A” may not be a fixed point, but may be a point where the axes 620 and 622 intersect. Accordingly, by adjusting the relative positions of the arms 624 and 626, the positions of the corresponding alignment members 602 and 604 may be altered. This movement may shift the common center of rotation “A”, but both axes 620 and 622 may continue to intersect the common center of rotation “A” as it is moved.

For example, if the knob 634 is moved to the right, the common center of rotation “A” may move to the left. Similarly, if the knob 634 is moved to the left, the common center of rotation “A” may move to the right. If the linkage assembly 606 is moved toward the distal ends of the alignment members 602 and 604, the common center of rotation “A” may shift towards the distal ends of the alignment members. If the linkage assembly 606 is moved toward the proximal ends of the alignment members 602 and 604, the common center of rotation “A” may shift away from the distal ends of the alignment members. As the center of rotation “A” is shifted, the arms 624 and 626 may maintain the alignment of the alignment members 602 and 604 with the center of rotation “A”.

In addition, the alignment instrument 600 may be designed to substantially align the dynamic stabilization systems 100A and 100B so that the axes of 154, 156, and 158 of pivots 144, 148, and 152, respectively, of the corresponding spinal stabilization devices 101A and 101B (FIG. 1B) point generally towards the common center of rotation “A”. In the present example, the linkage assembly 606 may be adjustable to accommodate variations in a distance “d” between polyaxial heads of the spinal stabilization devices 101A and 101B to which extensions may be secured as described with respect to FIG. 1B. Furthermore, the linkage assembly 606 may be adjusted to accommodate variations in an angle “α” between the two alignment members 602 and 604. The angle “α” may be the angle between axis 620 and axis 622 with respect to the common center of rotation “A.” The guide pin assembly 628 may also be substantially aligned with the common center of rotation “A” such that an axis 636 passing longitudinally through shaft 630 also passes through the common center of rotation “A”. The guide pin assembly 628 may be used to position the common center of rotation “A” by substantially aligning with anatomical landmarks on the patient.

In operation, the alignment members 602 and 604 may be coupled to the dynamic stabilization systems 100A and 100B while coupled to the linkage assembly 606. Alternatively, the alignment members 602 and 604 may be coupled to the dynamic stabilization systems 100A and 100B separately and then coupled to the linkage assembly 606.

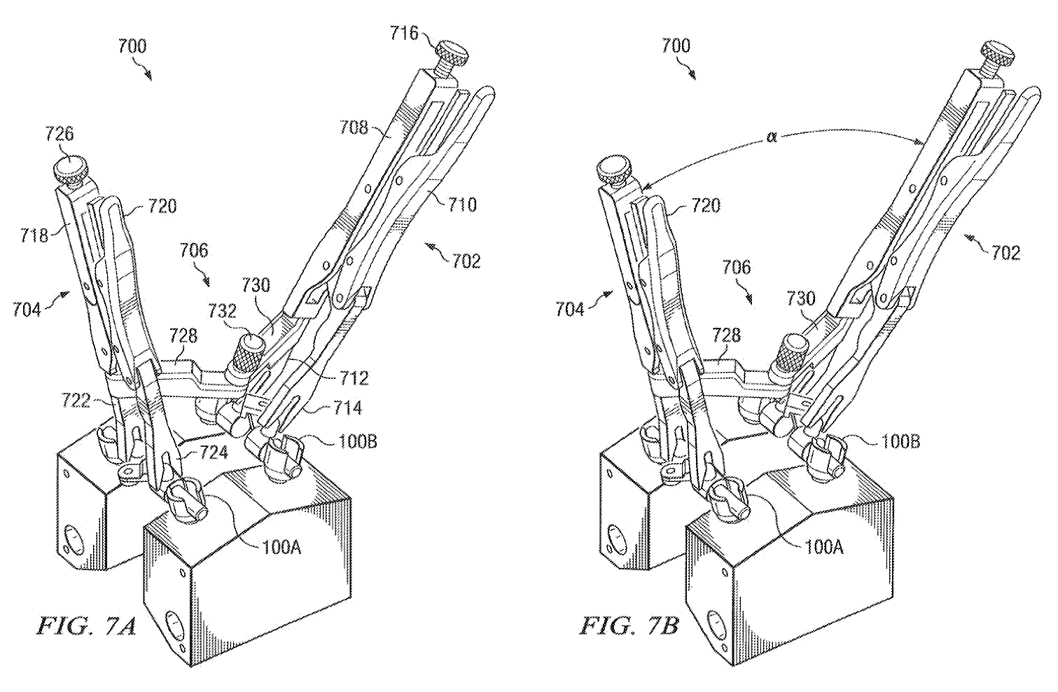

Referring to FIGS. 7A and 7B, in another embodiment, an alignment instrument 700 may be used to align one or more dynamic stabilization systems 100A and 100B (e.g., the spinal stabilization system 100 of FIG. 1B) to a center of rotation “A” (not shown). As described previously, portions of the dynamic stabilization systems 100A and 100B may be configured to rotate around the center of rotation “A”.

In the present example, the alignment instrument 700 may include adjustable gripping pliers 702 and 704 coupled by a linkage assembly 706. The adjustable gripping pliers 702 may include opposing handle portions 708 and 710 that may include couplers (e.g., a gripping means such as opposing jaws) 712 and 714, respectively, at a distal end thereof. The couplers 712 and 714 may be configured to couple to various features of the dynamic stabilization system 100B, including polyaxial heads (e.g., 106 and 108 of FIG. 1B), collars (e.g., 132 and 134 of FIG. 1B) and/or extensions (e.g., 126 and 128 of FIG. 1B). For example, the couplers 712 and 714 may include a yoke feature for snapping onto extensions 126 and 128. An adjustment screw 716 may be used to vary a distance between the couplers 712 and 714. The adjustable gripping pliers 704 may include opposing handle portions 718 and 720 that may include couplers (e.g., a gripping means such as opposing jaws) 722 and 724, respectively, at a distal end thereof. An adjustment screw 726 may be used to vary a distance between the couplers 722 and 724. The adjustable gripping pliers 702 and 704 may be designed to point to a common center of rotation (not shown), as has been described with respect to previous embodiments.

The linkage assembly 706 may include arms 728 and 730 that may be pivotally coupled to one another. The arm 730 may be coupled to the handle portion 708 (as illustrated) and/or to the handle portion 710, and arm 728 may be coupled to the handle portion 718 (as illustrated) and/or to the handle portion 720. The arms 728 and 730 may be coupled to the handle portions in such a way as to enable the linkage assembly 706 to rotate with respect to the adjustable gripping pliers 702 and 704. A guide pin assembly 732 may be used to adjust and align the linkage assembly 706. In the present example, linkage assembly 706 may be similar or identical to the linkage assembly 606 of FIG. 6 except for the manner in which the arms 728 and 730 are coupled to the handle portions.

The linkage assembly 706 may be adjustable to accommodate variations in a distance “d” (FIG. 7B) between polyaxial heads of dynamic stabilization systems 100A and 100B and to accommodate variations in an arc “α” between the adjustable gripping pliers 702 and 704. As described with respect to the linkage assembly 606 of FIG. 6, the linkage assembly 706 may enable the adjustable gripping pliers 702 and 704 to be moved while maintaining alignment of the adjustable gripping pliers and the corresponding dynamic stabilization systems 100B and 100A with the common center of rotation. The guide pin assembly 732 may also be substantially aligned with the common center of rotation and may be used to locate the common center of rotation by alignment of the guide pin assembly with anatomical landmarks using techniques such as fluoroscopy.

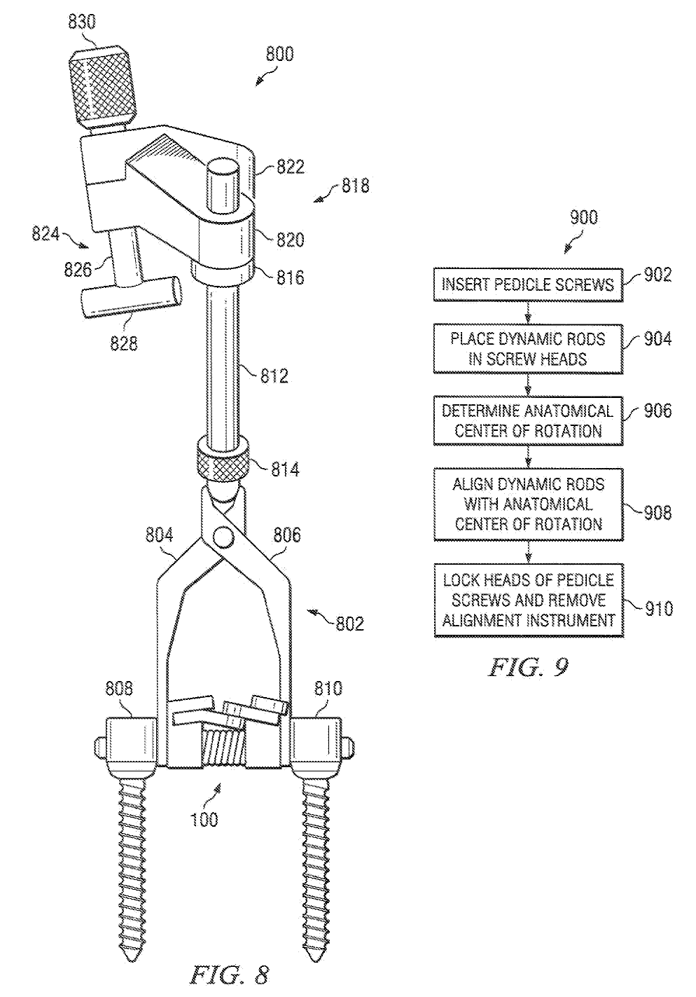

Referring to FIG. 8, in another embodiment, a portion 800 of an alignment instrument is illustrated. The alignment instrument may be used to align one or more dynamic stabilization systems (e.g., the spinal stabilization system 100 of FIG. 1B) to a center of rotation “A” (not shown). As described previously, portions of the dynamic stabilization system 100 may be configured to rotate around the center of rotation “A”.

The illustrated portion 800 of the alignment instrument may be coupled to the dynamic stabilization system 100 and another portion of the alignment instrument (not shown) may couple to another dynamic stabilization system. In the present example, the portion 800 may be configured to couple to various features of the dynamic stabilization system 100, including polyaxial heads (e.g., 106 and 108 of FIG. 1B), collars (e.g., 132 and 134 of FIG. 1B) and/or extensions (e.g., 126 and 128 of FIG. 1B).

The portion 800 may include adjustable gripping pliers 802 formed by opposing members 804 and 806. The opposing members 804 and 806 may include couplers 808 and 810, respectively, such as gripping jaws forming a yoke feature for snapping onto a feature of the dynamic stabilization system 100. The portion 800 may include a shaft 812 coupled to the gripping pliers 802. An adjuster 814 may be provided for varying a distance between the opposing members 804 and 806. For example, the adjuster 814 may be a knurled nut threaded onto a distal end of shaft 812 and coupled to the opposing members 804 and 806. The shaft 812 may further include a collar 816 that may be used to position the shaft with respect to a linkage assembly 818. The shaft 812 and/or opposing members 804 and 806 may be designed to point toward a common center of rotation that is also a common center of rotation for another portion (not shown) of the alignment instrument.

A linkage assembly 818 may include arms 820 and 822 that may be pivotally coupled to one another at a proximal end of each arm. The arm 820 may be coupled to shaft 812 and the arm 822 may be coupled to a similar shaft (not shown) of the alignment instrument. In the present example, a distal end of arm 820 may include a bore therethrough for receiving the proximal end of the shaft 812. The bore may have a longitudinal axis that may intersect the center of rotation.

The linkage assembly 818 may also include a guide pin assembly 824. The guide pin assembly 824 may include a shaft 826 having a foot 828 at a distal end thereof and a knob 830 at a proximal end thereof. The foot 828 may be placed proximal to or in contact with an outer tissue layer of a patient. The shaft 826 may be configured so that a longitudinal axis of the shaft may intersect the centre of rotation, and the shaft may be used for alignment purposes using, for example, fluoroscopy techniques. The knob 830 may be used to adjust the relative positions of the arms 820 and 822 and, accordingly, the corresponding shafts.

Linkage assembly 818 and the coupled shafts may be designed to point towards a common center of rotation. It is understood that the common center of rotation may not be a fixed point, but may be a point to where the shaft 812 and/or opposing members 804 and 806 are directed, as are corresponding components (not shown) coupled to arm 822. Accordingly, by adjusting the relative positions of the arms 820 and 822, the orientation of the corresponding shafts may be altered. This movement may shift the common center of rotation, but the design of the alignment instrument may ensure that the coupled dynamic stabilization systems may continue to intersect the common center of rotation “A” as the movement occurs.

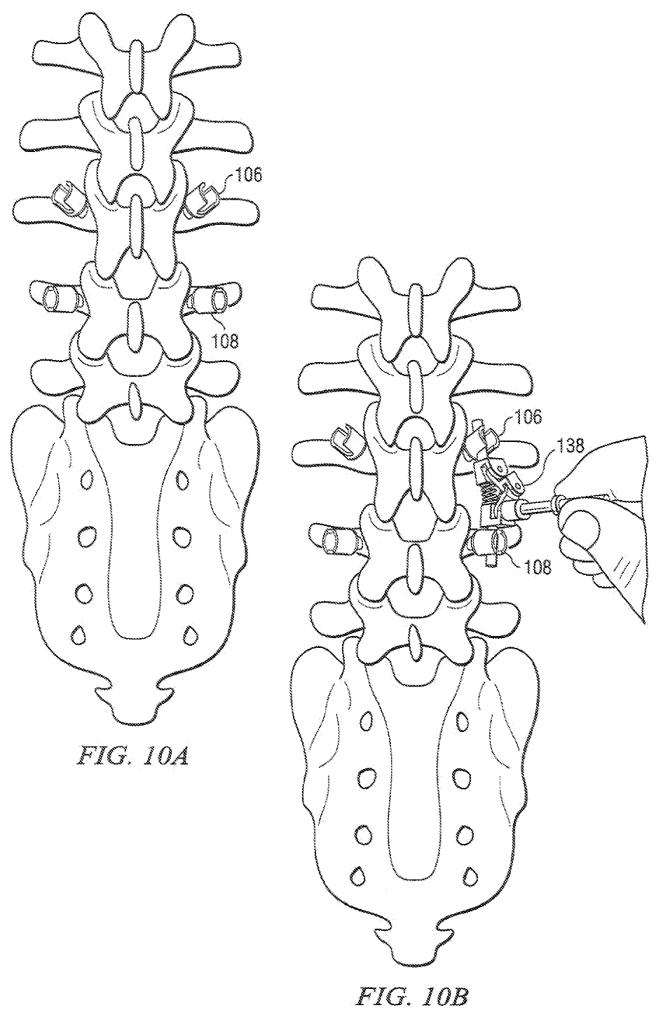

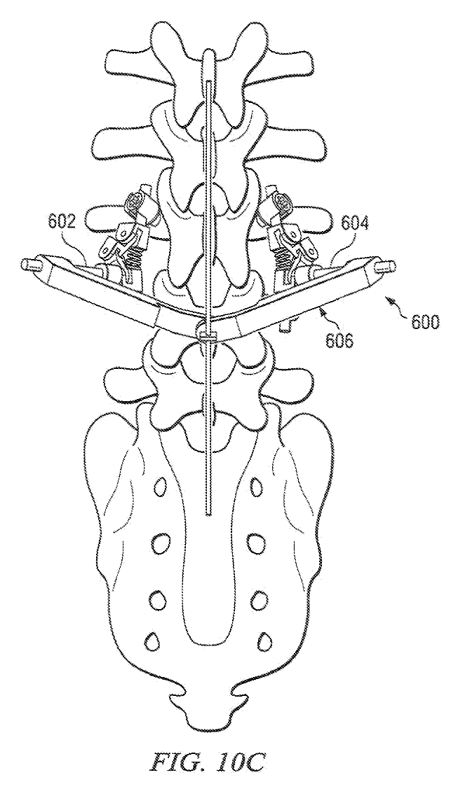

Referring to FIG. 9 and FIGS. 10A-10E, one embodiment of a method 900 is illustrated for substantially aligning one or more dynamic spinal stabilization systems (e.g., the dynamic stabilization system 100 of FIG. 1B) with a desired common center of rotation. It is understood that a location of the common center of rotation may be selected prior to or during a surgical procedure and may vary depending on such factors as a patient's particular spinal structure.

In step 902, a surgeon may insert bone anchors into vertebral bodies, as shown in FIG. 10A. The surgeon may then insert a spinal stabilization device (e.g., the spinal stabilization device 101 of FIG. 1B) into each pair of polyaxial heads corresponding to the bone anchors in step 904, as shown in FIG. 10B. In step 906, the surgeon may determine a desired center of rotation between the adjacent vertebral bodies. In certain embodiments, the surgeon may use a guide pin assembly coupled to an alignment instrument (e.g., the guide pin assembly 628 of the alignment instrument 600 of FIG. 6) to locate a midline of the patient or the sagittal plane, as shown in FIG. 10C. For example, the center of rotation may be located on the sagittal plane at the top plate of the lower vertebral body within the intervertebral space. In some embodiments, an alignment rod may be coupled to the alignment instrument to aid in positioning the alignment instrument as shown in FIG. 10C. In step 908, the surgeon may substantially align the dynamic stabilization systems with the center of rotation using an alignment instrument such as the alignment instrument 600 of FIG. 6, as shown in FIG. 10D. Subsequently, in step 910, the surgeon may lock the polyaxial heads and remove the alignment instrument, as shown in FIG. 10E.

It is understood that it may be desirable to maintain all of the polyaxial heads in the same plane. However, if the pedicles of adjacent vertebrae are out of alignment, it may be difficult to maintain alignment of the polyaxial heads because the vertebral bodies into which the bone anchors are embedded are not themselves properly aligned. Accordingly, the alignment instrument may be used to aid in compensating for the lack of alignment of adjacent vertebral bodies and may enable a surgeon to make a substantially rectangular box configuration of the polyaxial heads. As previously described, the alignment instrument may aid in orienting the dynamic stabilization systems to point toward a common center of rotation.

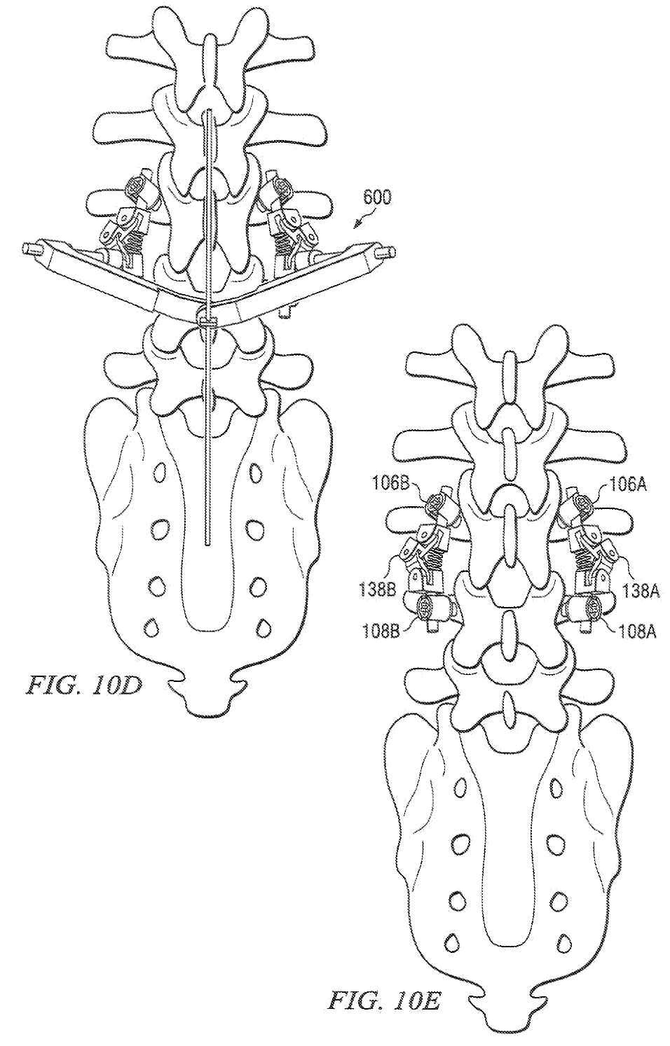

Referring to FIG. 11, in another embodiment, there is presented a method 1100 of substantially aligning one or more spherical motion dynamic spinal stabilization devices with an anatomical center of rotation is illustrated. In step 1102, a surgeon may attach an embodiment of a gripping tool to a dynamic stabilization rod or another component of the spherical motion dynamic spinal stabilization device. In step 1104, the surgeon may install one or more of the dynamic stabilization rods into polyaxial heads of pedicle anchor screws previously embedded into adjacent vertebral bodies. The surgeon, in step 1106, may then start locking caps into the ends of the polyaxial heads to hold the dynamic stabilization rods in place.

In step 1108, the surgeon may attach an alignment instrument (e.g., the alignment instrument 600 of FIG. 6) to the dynamic rod gripping tools. The surgeon may, in step 1110, adjust the dynamic rods to optimize the location of the dynamic rods with respect to the end plates of the vertebral bodies. In step 1112, the surgeon may rotate the alignment instrument to substantially match alignment features of the alignment instrument, such as a guide pin, with spinous processes on the vertebral bodies or other alignment indicators. In step 1114, the surgeon may tighten the locking caps to secure the dynamic rods in substantially proper alignment with the center of rotation between the two vertebral bodies, after which the alignment instrument may be detached from the dynamic rods and removed.

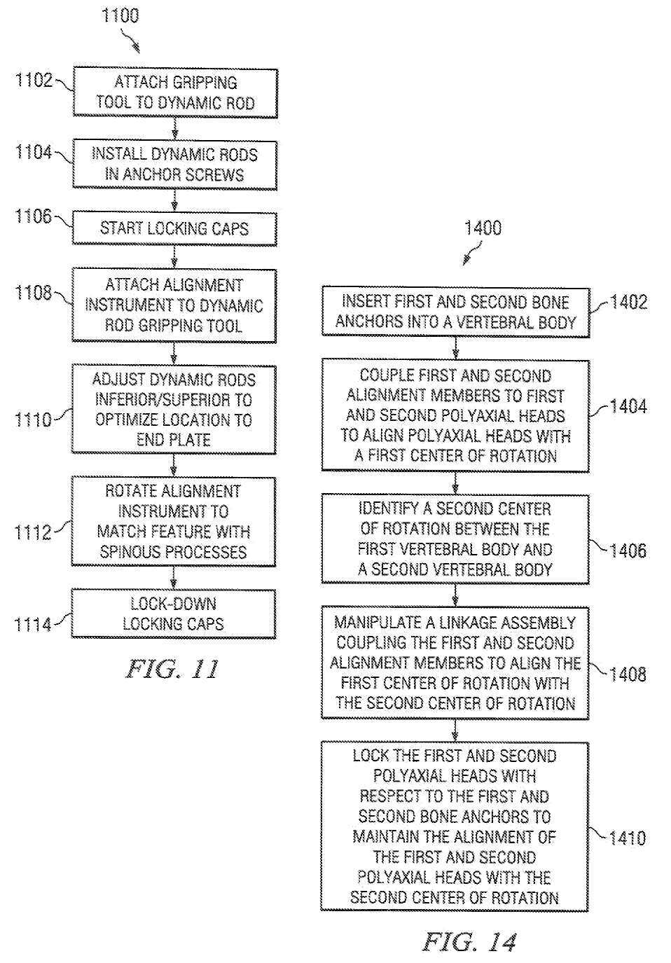

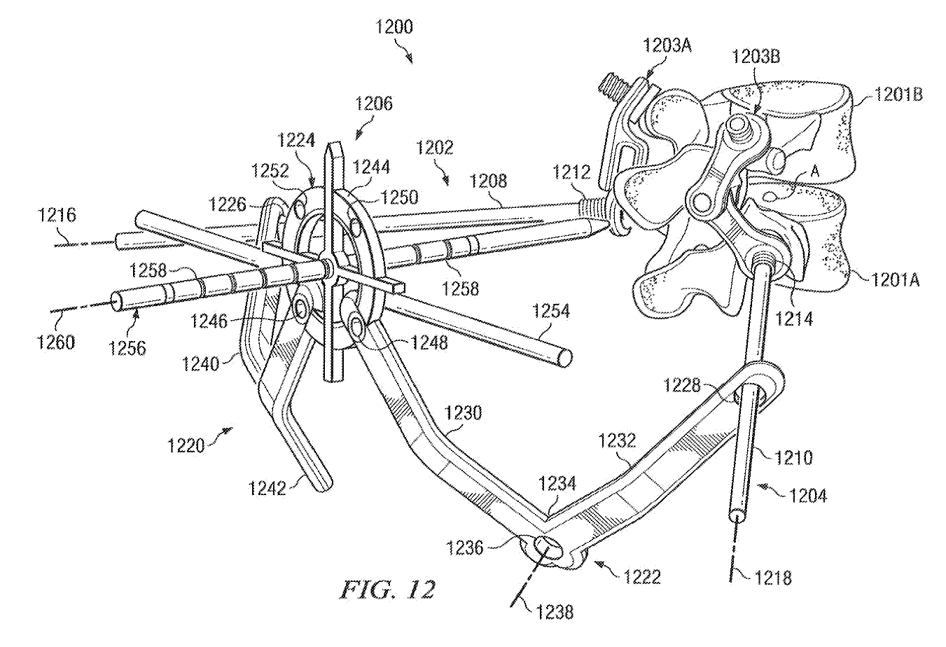

Referring to FIGS. 12 and 13, in another embodiment, an alignment instrument 1200 may be used to align one or more dynamic stabilization systems 1203A and 1203B to a center of rotation “A”. The dynamic stabilization systems 1203A and 1203B are conceptually similar to the dynamic stabilization systems 100A and 100b discussed previously. Additional detail on these systems may be found in the commonly assigned U.S. Provisional Application Ser. No. 60/786,898, entitled “FULL MOTION SPHERICAL LINKAGE IMPLANT SYSTEM,” filed Mar. 29, 2006. As described previously, portions of the dynamic stabilization system 1203 may be configured to rotate around the center of rotation “A”, which may be positioned between vertebral bodies 1201A and 1201B. As illustrated in FIGS. 12 and 13, the alignment instrument 1200 may attach to the dynamic stabilization systems 1203A and 1203B and may be used to alter the position of one or both of the dynamic stabilization systems prior to locking the dynamic stabilization systems into position.

In the present example, the alignment instrument 1200 may include alignment members 1202 and 1204 that may be coupled by a linkage assembly 1206. The alignment member 1202 may include a body portion 1208 (e.g., a shaft) and the alignment member 1204 may include a body portion 1210 (e.g., a shaft). Although not shown, each alignment member 1202 and 1204 may include a collar or other adjustment mechanism at a proximal end (relative to the linkage assembly 1206) of the respective shafts 1208 and 1210 for adjusting a position of the shafts with respect to the linkage assembly 1206.

The shafts 1208 and 1210 may include couplers 1212 and 1214, respectively, at the shafts' distal ends. Each coupler 1212 and 1214 may be configured to removably couple to a portion of the dynamic stabilization systems 1203A and 1203B, such as a polyaxial head. The couplers 1212 and 1214 may be separate components that attach to the shafts 1208 and 1210, or may be integrated into the distal ends of the shafts (e.g., the distal ends may be threaded to mate with the polyaxial heads or may be shaped to fit into a receptacle in a locking cap or other component that is coupled to the polyaxial head). The alignment members 1202 and 1204 may each have a longitudinal axis 1216 and 1218, respectively. In the present example, the longitudinal axes 1216 and 1218 may extend from the proximal end of each alignment member 1202 and 1204 to the distal ends, and may intersect the center of rotation “A”.

The linkage assembly 1206 may include arms 1220 and 1222 that may be coupled to a center portion 1224 of the linkage assembly at a proximal end of each arm. The arm 1220 may be coupled to alignment member 1202 and the arm 1222 may be coupled to alignment member 1204. In the present example, a distal end of each arm 1220 and 1222 may include a bore 1226 and 1228, respectively, for receiving the alignment members 1202 and 1204. The bores 1226 and 1228 may each have a longitudinal axis that may intersect the center of rotation “A”. For example, the axes of the bores 1226 and 1228 may coincide with the axes 1216 and 1218 of the alignment members 1202 and 1204. It is understood that although the bores 1226 and 1228 are illustrated in FIG. 12 as being relatively wide compared to the shafts 1208 and 1210, the bores may be sized to receive the shafts in a relatively tight fit while still allowing movement (e.g., rotation) of the shafts within the bores. The collars (not shown) or other adjustment mechanisms may prevent the arms 1220 and 1222 from movement in the direction of the distal ends of the alignment members 1202 and 1204.

In the present example, the arm 1222 may include a first portion 1230 and a second portion 1232. Each portion 1230 and 1232 may be bent or curved to enable the bore 1228 to point towards the center of rotation “A”. The portions 1230 and 1232 may be coupled at an elbow 1234. The elbow 1234 may include a bore 1236 positioned so that a longitudinal axis 1238 extending through the bore may intersect the center of rotation “A”. In some examples, the bore 1236 may be configured to receive an alignment member or other tool. It is understood that the bore 1236 may not be present in some embodiments. Although shown with the two portions 1230 and 1232, it is understood that the arm 1222 may be formed as a single member having varying shapes (e.g., curved or bent). In other embodiments, the arm 1222 may be relatively straight, and bore 1228 may be formed to adjust for the lack of curvature of the arm. The arm 1220 may include a first portion 1240 and a second portion 1242. Although not shown in detail in FIG. 12, the arm 1220 may include features that are similar or identical to features of the arm 1222 discussed previously.

In the present example, the center portion 1224 of the linkage assembly 1206 may include a guide mechanism. In the illustrated embodiment, the guide mechanism may be a wheel-like member having an outer ring 1244. The outer ring 1244 may serve as an alignment mechanism (e.g., an alignment cross) to aid in alignment using fluoroscopy or another suitable imaging process. It is understood that other shapes are possible and that the center portion 1224 is not limited to the shape illustrated in FIG. 12. The arms 1220 and 1222 may couple to the outer ring 1244. The couplings may be fixed or movable (e.g., pivotal) relative to the linkage assembly depending on the particular configuration of the alignment instrument 1200. In the present embodiment, the outer ring 1244 may include coupling points (e.g., bores or other attachment means) 1246, 1248, 1250, and 1252. In some embodiments, additional arms (not shown) may be coupled to the center portion 1224. For example, arms may be coupled to the outer ring 1244 at connection points 1250 and 1252. In such embodiments, alignment members (not shown) coupled to the arms may be used to align additional polyaxial heads. The arms may be coupled to the center portion 1224 at the same time as the arms 1220 and 1222, or at different times (e.g., at an earlier or later time in a surgical procedure). Although the connection points 1246 and 1248 are illustrated as being equidistant from a vertical axis (not shown) dividing the outer ring 1244 into left and right halves, it is understood that the connection points may not be equidistant in some embodiments.

An adjustment mechanism (e.g., a rod) 1254 may be used to manipulate the alignment instrument 1200. For example, the rod 1254 may be used to adjust the center portion 1224 in the cephalad/caudal and/or anterior/posterior directions, thereby moving the center of rotation “A”.

A shaft 1256 may be used to aid in alignment. For example, the shaft 1256 may include various markings 1258. The markings 1258 may indicate a distance of the shaft's distal end from the center of rotation “A”. For example, if the distal end of the shaft 1256 is touching the patient's skin and if a marking 1258 labeled “10 cm” is adjacent to the proximal surface of the center portion 1224, then the tip of the shaft 1256 may be ten centimeters from the point of rotation “A” (e.g., the center of rotation “A” is approximately ten centimeters under the patient's skin). Accordingly, the shaft 1256 may provide a visual guide to the depth of the center of rotation “A”. In the present example, the shaft 1256 may have a longitudinal axis 1260 that intersects the center of rotation “A”. Accordingly, the shaft 1256 may be used as an alignment guide using fluoroscopy or another suitable imaging technique.

As described previously, linkage assembly 1206 and corresponding alignment members 1202 and 1204 may be designed to point towards the common center of rotation “A”. It is understood that the common center of rotation “A” may not be a fixed point, but may be a point where the axes 1216 and 1218 intersect. Accordingly, by adjusting the relative positions of the arms 1220 and 1222, the positions of the corresponding alignment members 1202 and 1204 may be altered. This movement may shift the common center of rotation “A”, but both axes 1216 and 1218 may continue to intersect the common center of rotation “A” as it is moved.

In the present example, if the rod 1254 is moved to the right, the common center of rotation “A” may move to the right. Similarly, if the rod 1254 is moved to the left, the common center of rotation “A” may move to the left. If the rod 1254 is moved inward (e.g., towards the polyaxial heads), the common center of rotation “A” may shift inward. If the rod 1254 is moved outward (e.g., away from the polyaxial heads), the common center of rotation “A” may shift outward. As the center of rotation “A” is shifted, the arms 1220 and 1222 may maintain the alignment of the alignment members 1202 and 1204 with the center of rotation “A”. Accordingly, the center of rotation “A” of the polyaxial heads may be aligned with a desired center of rotation using the alignment instrument 1200.

In operation, the alignment members 1202 and 1204 may be coupled to the dynamic stabilization systems 1203A and 1203B while coupled to the linkage assembly 1206. Alternatively, the alignment members 1202 and 1204 may be coupled to the dynamic stabilization systems 1203A and 1203B separately and then coupled to the linkage assembly 1206.

It is understood that the alignment instrument 1200 may be modified to provide similar or identical functionality in a different configuration. For example, rather than being bores having a fixed axis, the bores 1226 and 1228 may be modified to provide an adjustable alignment mechanism (e.g., an adjustable housing and/or locking mechanism). Furthermore, the alignment members 1202 and 1206 may be integrated with the arms 1220 and 1222, and/or the alignment members may couple to the arms using a different coupling mechanism than the illustrated bores 1226 and 1228. It is also understood that the alignment members 1202 and 1204 need not be shafts, but may have other shapes.

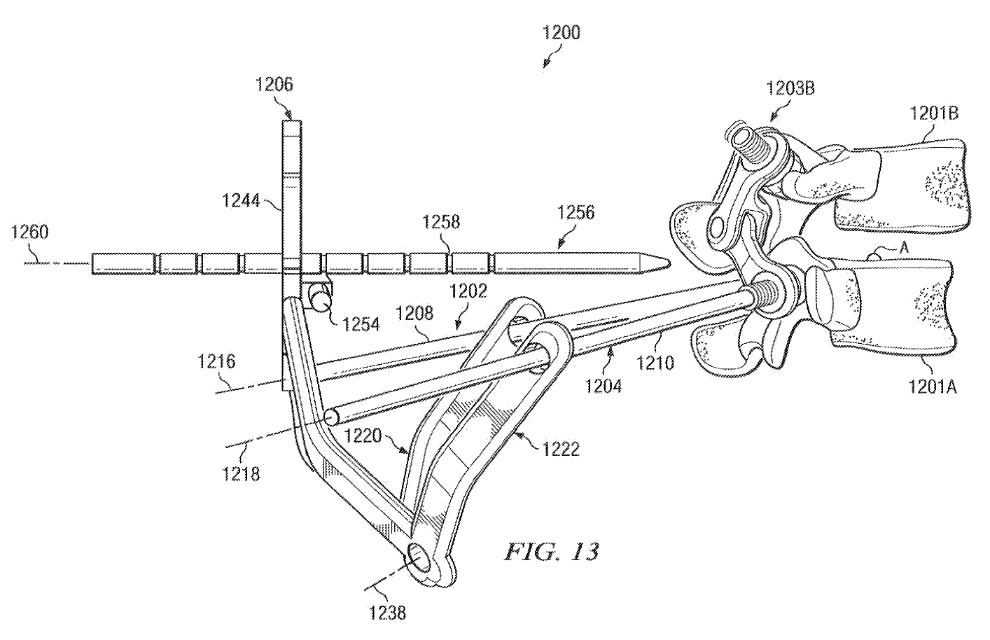

Referring to FIG. 14, in another embodiment, a method 1400 may be used to align a dynamic spinal stabilization system. The method 1400 may be used, for example, with the alignment instrument 600 of FIG. 6 or the alignment instrument 1200 of FIG. 12.

In step 1402, first and second bone anchors may be inserted into a vertebral body. The first and second bone anchors may include first and second polyaxial heads, respectively, as described previously. It is understood that while a single vertebral body is used for purposes of example, the first and second bone anchors may be inserted into separate vertebral bodies. In step 1404, first and second alignment members may be coupled to the first and second polyaxial heads, respectively, where the first and second alignment members are automatically centered on a first center of rotation. In step 1406, a second center of rotation may be identified between the first vertebral body and a second vertebral body. It is understood that the second center of rotation may be identified prior to step 1402. In step 1408, a linkage assembly coupling the first and second alignment members may be manipulated to align the first center of rotation with the second center of rotation, where the first and second polyaxial heads are thereby aligned with the second center of rotation. In step 1410, the first and second polyaxial heads may be locked with respect to the first and second bone anchors to maintain the alignment of the first and second polyaxial heads with the second center of rotation.

Although only a few exemplary embodiments of this disclosure have been described in detail above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

What is claimed is:

1. An alignment instrument comprising:
a first alignment member having a first body portion connecting a first proximal end and a first distal end, wherein the first distal end is configured to couple to a spinal stabilization system at a first point, and a first longitudinal axis extending through the first body portion and intersecting a center of rotation located distally from the first distal end;
a second alignment member having a second body portion connecting a second proximal end and a second distal end, wherein the second distal end is configured to couple to the spinal stabilization system at a second point, and a second longitudinal axis extending through the second body portion and intersecting the center of rotation located distally from the second distal end; and
a linkage assembly having a first arm rotatably coupled to the first alignment member and a second arm rotatably coupled to the second alignment member.

2. The alignment instrument of claim 1 wherein the first arm includes a bore formed therethrough for receiving the first alignment member, wherein a longitudinal axis of the first arm's bore intersects the center of rotation.

3. The alignment instrument of claim 2 wherein the second arm includes a bore formed therethrough for receiving the second alignment member, wherein a longitudinal axis of the second arm's bore intersects the center of rotation.

4. The alignment instrument of claim 1 further comprising a guide pin assembly coupled to the linkage assembly, wherein the guide pin assembly includes a shaft extending towards the center of rotation and having a longitudinal axis intersecting the center of rotation.

5. The alignment instrument of claim 4 wherein the guide pin assembly further comprises a foot coupled to a distal end of the shaft.

6. The alignment instrument of claim 1 wherein the first and second distal ends are configured to couple to first and second polyaxial heads, respectively, of the spinal stabilization system.

7. The alignment instrument of claim 1 wherein the first and second distal ends are configured to couple to first and second rod portions, respectively, of the spinal stabilization system.

8. The alignment instrument of claim 1 wherein the first body portion includes a first collar positioned between the first arm and the first distal end.

9. The alignment instrument of claim 8 wherein the second body portion includes a second collar positioned between the second arm and the second distal end.

10. The alignment instrument of claim 8 wherein the first collar is adjustable along the first longitudinal axis.

11. The alignment instrument of claim 1 further comprising adjustment means for adjusting a position of the first arm relative to the second arm.

12. The alignment instrument of claim 11 wherein the first and second longitudinal axes intersect the center of rotation regardless of the relative positions of the first and second arms.

13. The alignment instrument of claim 1 wherein the linkage assembly includes a shaft extending towards the center of rotation, wherein the shaft has a longitudinal axis intersecting the center of rotation.

14. The alignment instrument of claim 13 further comprising first and second bores formed in a proximal end of the first and second arms, respectively, wherein the first and second bores receive the shaft and rotatably couple the first and second arms to the shaft.

15. The alignment instrument of claim 1 wherein the linkage assembly is configured to move the center of rotation in a first direction when the linkage assembly is moved in a second direction that is opposite the first direction.

16. The alignment instrument of claim 1 wherein the linkage assembly is configured to move the center of rotation in a first direction when the linkage assembly is moved in the first direction.

17. The alignment instrument of claim 1 wherein the linkage assembly includes a central portion coupled to the first and second arms.

18. The alignment instrument of claim 17 wherein the central portion includes a first ring, wherein the first and second arms are coupled to the first ring.

19. The alignment instrument of claim 18 wherein the first and second arms are pivotally coupled to the first ring.

20. The alignment instrument of claim 19 wherein the first and second arms are coupled to the first ring equidistantly from a vertical axis dividing the first ring into equal halves.

21. The alignment instrument of claim 17 wherein the central portion includes a second ring that is concentric to and located within the first ring.

22. The alignment instrument of claim 21 further comprising a plurality of members coupling the first and second rings.

23. The alignment instrument of claim 17 wherein the first arm includes first and second portions coupled at a joint, wherein an axis substantially perpendicular to a surface of the joint facing the center of rotation intersects the center of rotation.

24. The alignment instrument of claim 17 further comprising a shaft extending through the central portion, wherein a longitudinal axis of the shaft is aligned with the center of rotation.

25. An alignment instrument comprising:
a first alignment member configured to couple to a dynamic spinal stabilization system, wherein the first alignment member includes a first longitudinal axis that intersects a center of rotation located distally from the first alignment member;
a second alignment member configured to couple to the spinal stabilization system, wherein the second alignment member includes a second longitudinal axis that intersects the center of rotation located distally from the second alignment member; and
a linkage assembly having a first arm coupled to the first alignment member and a second arm coupled to the second alignment member, wherein linkage assembly is configured to maintain the alignment of the first and second longitudinal axes with the center of rotation during movement of the first and second arms.

26. The alignment instrument of claim 25 wherein a distal end of the first alignment member is configured to couple to a polyaxial head of the spinal stabilization system, and wherein a vertical axis of the polyaxial head is aligned with the center of rotation when coupled to the first alignment member.

27. The alignment instrument of claim 25 wherein the first and second arms each include an attachment mechanism for releasably coupling to the first and second alignment members, respectively.

28. The alignment instrument of claim 27 wherein the attachment mechanism includes a bore sized to receive one of the first and second alignment members.

29. The alignment instrument of claim 25 further comprising a shaft extending through the linkage assembly so that a longitudinal axis of the shaft intersects the center of rotation.

30. The alignment instrument of claim 29 wherein the shaft includes a plurality of external markings representing a distance of a distal end of the shaft from the center of rotation.

31. A method for aligning a dynamic spinal stabilization system comprising:
inserting first and second bone anchors into a first vertebral body, wherein the first and second bone anchors include first and second polyaxial heads, respectively;
coupling first and second alignment members to the first and second polyaxial heads, respectively, wherein the first and second alignment members are automatically centered on a first center of rotation;
identifying a second center of rotation between the first vertebral body and a second vertebral body;
manipulating a linkage assembly coupling the first and second alignment members to align the first center of rotation with the second center of rotation, wherein the first and second polyaxial heads are thereby aligned with the second center of rotation; and
locking the first and second polyaxial heads with respect to the first and second bone anchors to maintain the alignment of the first and second polyaxial heads with the second center of rotation.

32. The method of claim 31 further comprising inserting third and fourth bone anchors into the second vertebral body, wherein the third and fourth bone anchors include third and fourth polyaxial heads, respectively.

33. The method of claim 32 further comprising:
coupling the first and second alignment members to the third and fourth polyaxial heads, respectively;
manipulating the linkage assembly coupling the first and second alignment members to align the third and fourth polyaxial heads with the second center of rotation; and
locking the third and fourth polyaxial heads with respect to the third and fourth bone anchors to maintain the alignment of the third and fourth polyaxial heads with the second center of rotation.

34. The method of claim 32 further comprising:
coupling third and fourth alignment members to the third and fourth polyaxial heads, respectively, wherein the third and fourth alignment members are automatically centered on the first center of rotation;
manipulating the linkage assembly coupling the third and fourth alignment members to align the first center of rotation with the second center of rotation, wherein the third and fourth polyaxial heads are thereby aligned with the second center of rotation; and
locking the third and fourth polyaxial heads with respect to the third and fourth bone anchors to maintain the alignment of the third and fourth polyaxial heads with the second center of rotation.

35. The method of claim 34 wherein manipulating the linkage assembly to align the first and second polyaxial heads with the second center of rotation occurs simultaneously with manipulating the linkage assembly to align the third and fourth polyaxial heads with the second center of rotation.

36. The method of claim 32 further comprising inserting a first stabilization member into the first and third polyaxial heads and inserting a second stabilization member into the second and fourth polyaxial heads.

37. The method of claim 31 wherein manipulating the linkage assembly includes moving the linkage assembly in a first direction to move the first center of rotation in a second direction opposite the first direction.

38. The method of claim 31 wherein manipulating the linkage assembly includes moving the linkage assembly in a first direction to move the first center of rotation in the first direction.
